(19) United States
(12) Patent Application Publication
(10) Pub. No.: US 2021/0060327 A1
(43) Pub. Date: Mar. 4, 2021

(54) PERCUTANEOUS ACCESS DEVICE

(71) Applicants: Paul Singleton, Vandalia, OH (US); Kevin Pruszynski, Dayton, OH (US)

(72) Inventors: Paul Singleton, Vandalia, OH (US); Kevin Pruszynski, Dayton, OH (US)

(21) Appl. No.: 17/006,999

(22) Filed: Aug. 31, 2020

Related U.S. Application Data
(60) Provisional application No. 62/893,371, filed on Aug. 29, 2019.

(51) Int. Cl. A61M 39/02

(52) U.S. Cl. CPC A61M 39/0247; A61M 1/3655

(57) ABSTRACT

A percutaneous access device comprises a frame, a hatch and a hinge that couples the frame to the hatch. The frame has a passageway that extends therethrough. When the percutaneous access device is implanted in a patient, the frame is positioned beneath a layer of skin of the patient against a bodily vessel. Moreover, the hinge biases the hatch closed against the frame in a normally closed position such that liquid flowing through a lumen of the vessel cannot escape through the percutaneous access device. However, the application of force against the hatch from an object (such as a needle) that extends through the passageway causes the hatch to open outwardly from the frame and into the lumen of the vessel. Correspondingly, the hinge biases the hatch back to the normally closed position upon removal of the object.

### CROSS REFERENCE TO RELATED APPLICATIONS

This application claims the benefit of U.S. Provisional Patent Application Ser. No. 62/893,371, filed Aug. 29, 2019, entitled PERCUTANEOUS ACCESS DEVICE, the disclosure of which is hereby incorporated herein by reference.

### BACKGROUND

Various aspects of the present disclosure relate generally to a medical device and more particularly to a percutaneous access device for access to a bodily vessel.

There are occasions where it becomes medically necessary to inject a substance into, or remove a substance from, a bodily vessel. By way of example, should a person's kidneys become unable to filter waste products from their blood, toxins will eventually build up in the bloodstream causing a high risk of medical complications. However, hemodialysis (often referred to generally, as dialysis) can be used to artificially remove the toxins from the bloodstream. When a patient undergoes dialysis, a machine pumps blood out of the patient's body through an artificial kidney (dialyzer) that filters out waste. The cleaned blood is then returned to the patient's body.

In order to pump the patient's blood through the dialyzer, vascular access is required. Many patients that require dialysis for extended periods of time utilize a self-cannulation method referred to as a “buttonhole technique” for vascular access. The buttonhole technique allows the patient to insert the dialysis needles into the same spot each time dialysis is performed. In this regard, vascular access using the buttonhole technique requires an arteriovenous (AV) fistula or an AVs graft for vascular access. The AV fistula is created by directly connecting a person's artery and vein. The AV graft is similar in many respects to the AV fistula, except that a tube is utilized to surgically connect an artery to a vein.

### BRIEF SUMMARY

According to aspects of the present disclosure, a percutaneous access device comprises a frame, a hatch, and a hinge that couples the frame to the hatch. The frame has a passageway that extends therethrough. Moreover, the hinge couples the frame to the hatch such that the hatch is biased in a normally closed position against the frame.

When the percutaneous access device is implanted in a patient, the hatch, hinge, and frame define an access port. More particularly, the frame is positioned beneath a layer of skin of the patient, against a bodily vessel. Moreover, the hinge biases the hatch closed against the frame in a normally closed position such that liquid flowing through a lumen of the vessel cannot escape through the percutaneous access device. However, the application of force against the hatch from an object (such as a needle) causes the hatch to open outwardly from the frame, allowing the object to extend through the passageway and pass into the lumen of the vessel (thus, the hatch opens inwardly relative to internal volume of the vessel). Correspondingly, the hinge automatically biases the hatch back to the normally closed position upon removal of the object.

According to further aspects of the present disclosure herein, a percutaneous access device comprises an access port and a sleeve (e.g., biomaterial expanded polytetrafluoroethylene ePTFE) defining a body, where the access port is attached to the body of the sleeve. The access port includes a frame, a hatch, and a hinge that couples the frame to the hatch. The frame has a passageway that extends therethrough. Moreover, the hinge couples the frame to the hatch such that the hatch is biased in a normally closed position against the frame.

When the percutaneous access device implanted in a patient (e.g., with an AV fistula or AV graft), the frame is positioned beneath a layer of skin of the patient against a bodily vessel, and the sleeve extends over the vessel (e.g., the sleeve extends to both sides of the frame along a length of the vessel). The hinge biases the hatch closed against the frame in a normally closed position such that liquid flowing through a lumen of the vessel cannot escape through the percutaneous access device. However, the application of force against the hatch from an object (such as a needle) causes the hatch to open outwardly from the frame and inwardly towards the sleeve, allowing the object to extend through the passageway and pass into the lumen of the vessel and/or into an interior volume of the sleeve. Correspondingly, the hinge biases the hatch back to the normally closed position upon removal of the object.

According to yet further aspects of the present disclosure, a percutaneous access device is provided. The percutaneous access device comprises a body having a neck that transitions into a shoulder. A first insert extends from a first end of the shoulder and a second insert extends from a second end of the shoulder. In this regard, a first passageway extends through the first insert, through the shoulder, and through the second insert. A second passageway extends through the neck and into the first passageway. The percutaneous access device also includes a frame having a portal opening defining a passageway therethrough, where the frame is positioned in-line with the neck. Here, the frame can be separate from the neck or integral or otherwise defined by at least a portion of the neck. The percutaneous access device also includes a hatch, and a hinge that couples the frame to the hatch such that the hatch is biased in a normally closed position against the frame.

When the percutaneous access device implanted in a patient, the frame is positioned beneath a layer of skin of the patient, and the body in inserted in-line with a vessel such that the vessel extends over the first insert and the second insert. Here, the first passageway is thus in-line with a lumen of the vessel. Also, the hatch is biased closed against the frame in a normally closed position such that liquid flowing through the lumen of the vessel cannot escape through the percutaneous access device. However, the application of force against the hatch from an object outside the skin (such as a needle) causes the hatch to open outwardly from the frame and inwardly towards the body, allowing the object to pass through the second passageway and into the first passageway. As such, at least a tip of the object has access into the lumen of the vessel and/or the first passageway of the body. Likewise, the hatch biases back to the normally closed position upon removal of the object.

According to still further aspects of the present disclosure, a percutaneous access device is provided. The percutaneous access device comprises a body having a first neck that transitions into a shoulder, and a second neck that transitions into the shoulder. A first insert extends from a first end of the shoulder and a second insert extends from a second end of the shoulder. In this regard, a first passageway extends through the first insert, through the shoulder, and through the second insert. A second passageway extends through the first neck and into the first passageway. A third passageway extends through the second neck and into the first passageway. The percutaneous access device also includes a first frame having a portal opening defining a passageway therethrough, where the first frame is positioned in-line with the first neck. Here, the first frame can be separate from the first neck or integral or otherwise defined by at least a portion of the first neck. The percutaneous access device also includes a first hatch, and a first hinge that couples the first frame to the first hatch. In a practical application, the first hatch is biased in a normally closed position against the first frame. Analogously, the percutaneous access device includes a second frame having a portal opening defining a passageway therethrough, where the second frame is positioned in-line with the second neck. Here, the second frame can be separate from the second neck or integral or otherwise defined by at least a portion of the second neck. The percutaneous access device also includes a second hatch, and a second hinge that couples the second frame to the second hatch. Analogous to that above, the second hatch can be biased in a normally closed position against the second frame.

When the percutaneous access device implanted in a patient, the first frame and the second frame are positioned beneath a layer of skin of the patient, and the body in inserted in-line with a vessel such that the vessel extends over the first insert and the second insert. Here, the first passageway is thus in-line with a lumen of the vessel. Also, the first hatch and the second hatch are each in a normally closed position such that liquid flowing through the lumen of the vessel cannot escape through the percutaneous access device. However, the application of force against the first hatch from a first object outside the skin (such as a needle) causes the first hatch to open outwardly from the first frame and inwardly towards the body, allowing the first object to pass through the second passageway and into the first passageway. As such, at least a tip of the first object has access into the lumen of the vessel and/or the first passageway of the body. Likewise, the first hatch biases back to the normally closed position upon removal of the first object.

Analogously, the application of force against the second hatch from a second object outside the skin (such as a needle) causes the second hatch to open outwardly from the second frame and inwardly towards the body, allowing the second object to pass through the third passageway and into the first passageway. As such, at least a tip of the second object has access into the lumen of the vessel and/or the first passageway of the body. Likewise, the second hatch biases back to the normally closed position upon removal of the second object. The first hatch and second hatch can be operable independent of each other.

### BRIEF DESCRIPTION OF THE SEVERAL VIEWS OF THE DRAWINGS

FIG. 1 is a cross-section illustration of a percutaneous access device surgically implanted in a bodily vessel, according to aspects of the present disclosure herein;

FIG. 2 is a cross-section illustration of a needle inserted through the percutaneous access device of FIG. 1, according to aspects of the present disclosure;

FIG. 3 is a perspective illustration of a percutaneous access device according to aspects of the present disclosure;

FIG. 4 is a cross-section illustration of a percutaneous access device including a sleeve, which is surgically implanted in a bodily vessel, according to aspects of the present disclosure herein;

FIG. 5 is a cross-section illustration of a needle inserted through the percutaneous access device of FIG. 4, according to aspects of the present disclosure;

FIG. 6 is another example percutaneous access device according to further aspects of the present disclosure;

FIG. 7 is a top view of the percutaneous access device of FIG. 6;

FIG. 8 is yet another example percutaneous access device according to further aspects of the present disclosure;

FIG. 9 is an example hatch that can be utilized with any of the percutaneous access device configurations described more fully herein;

FIG. 10 is still another example percutaneous access device according to further aspects of the present disclosure;

FIG. 11 is a view illustrating a top partial view of a percutaneous access device and a corresponding bottom view of a mating needle alignment fixture illustrating a magnet configuration;

FIG. 12 is a schematic illustration of the percutaneous access device of FIG. 8 installed in a vessel of a patient's arm, further illustrating a needle alignment fixture used for aligning an aperture in the needle alignment fixture with the hatch of the percutaneous access device, according to aspects of the present disclosure;

FIG. 13 is a view illustrating an example configuration where the needle alignment fixture is readily attached to, and separated from a percutaneous access device according to aspects of the present disclosure;

FIG. 14 illustrates an example two-hatch percutaneous access device according to aspects of the present disclosure; and

FIG. 15 is a view illustrating a top partial view of a percutaneous access device and a corresponding bottom view of a mating needle alignment fixture illustrating a magnet configuration.

### DETAILED DESCRIPTION

Aspects of the present disclosure are directed to a percutaneous access device that can function as a “buttonhole” port for self-cannulation. More particularly, the percutaneous access device as described more fully herein, facilitates the ability to insert an object such as a needle into a bodily vessel at the same location, in a consistent and repeatable manner. As used herein, a vessel is a tube in the body that carries fluid, e.g., artery, vein, etc. A lumen is the interior volume of the vessel.

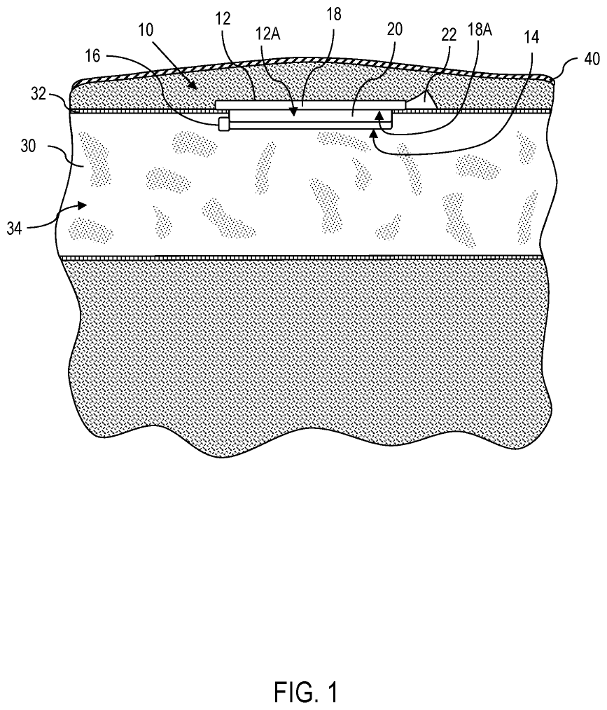

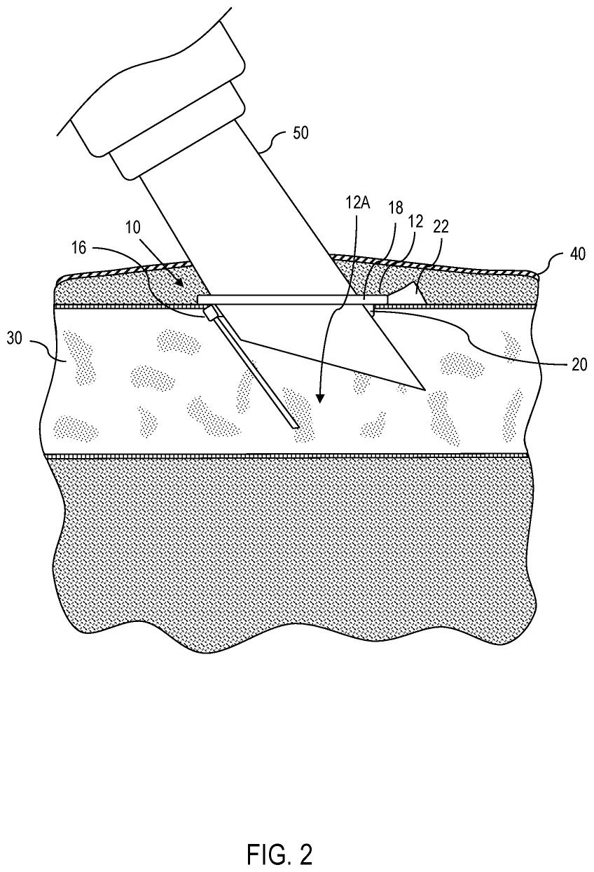

Referring now to the drawings, and in particular to FIG. 1 and FIG. 2, an example percutaneous access device 10 is illustrated. The percutaneous access device 10 includes in general, a frame 12, a hatch 14, and a hinge 16 that couples the frame 12 to the hatch 14. A passageway 12A extends through the frame 12, which, in cooperation with the hatch 14, defines an access port that enables an object to enter from outside of the percutaneous access device 10 and pass through the frame 12. As illustrated, the frame 12 includes a base member 18 having a first major surface 18A. An extension 20 defines a tube that extends or otherwise flanges from the base member 18 in the direction of the first major surface 18A. The frame 12 can take on other configurations, some examples of which are described in greater detail herein.

The percutaneous access device 10 may also optionally include an alignment protrusion 22. As illustrated, the alignment protrusion 22 extends from the frame 12 (e.g., the base member 18 in a direction opposite the first major surface 18A). The alignment protrusion 22 indicates a known position beneath the skin relative to the passageway 12A when the percutaneous access device 10 is implanted in the patient. In this regard, the alignment protrusion 22 can be any raised member. Moreover, the alignment protrusion 22 can be located in other positions so as to provide an indication to the user as to where to locate the hatch 14.

The percutaneous access device 10 is intended to be implanted in a patient. For instance, in the illustrative implementation, the percutaneous access device 10 is attached to a bodily vessel 30. For instance, sutures can be stitched around the frame 12, e.g., along the perimeter of the base member 18, to secure the percutaneous access device 10 against a wall 32 of the vessel 30. More particularly, in this illustrative implementation, the frame 12 is positioned beneath a layer of skin of the patient such that the first major surface 18A of the base member 18 is against the outside of the vessel wall 32, and the base member 18 is sutured or otherwise secured to the vessel 30. The extension 20 in this example, functions as a neck that transitions through the vessel wall 32, allowing the base member 18 to be positioned exterior to the vessel wall 32 and the extension 20 to neck into the vessel 30. In this manner, the passageway 12A extends through the base member 18 and the extension 20, and thus passes through the vessel 30.

As illustrated, the extension 20 allows the hatch 14 to be positioned within the vessel 30, i.e., interior to the vessel wall 32. For instance, as illustrated, the hatch 14 is positioned proximate to an inside surface of the wall 32 of the vessel 30. Here, the hinge 16 biases the hatch 14 closed against the frame 12 in a normally closed position such that liquid flowing through a lumen of the vessel 30 cannot escape through the percutaneous access device 10. In practice, the frame 12 can take on other shapes and configurations. Moreover, the hatch 14 can be positioned anywhere along the base member 18 and/or extension 20. Thus in other embodiments, the hatch 14 is coupled to the frame 12 by the hinge 16 at a position exterior to the vessel 30, e.g., within the extension 20, or adjacent to the vessel wall 32 within the extension 20, etc.

In a practical application, such as for dialysis, chemotherapy or other recurring medical treatment, the percutaneous access device 10 may be implanted in the arm or other suitable location of the patient. When the percutaneous access device 10 implanted in a patient, the base member 18 of the frame 12 is positioned beneath a layer of skin 40 of the patient. The first major surface 18A of the base member 18 faces the bodily vessel 30, and the extension 20 passes through the wall 32 of the bodily vessel 30. For instance, in a graft, the extension 20 may run between two sections of a cut vessel. However, this is still considered “passing through” because the port 10 provides access to a lumen 34 of the vessel 30.

With specific reference to FIG. 1, as noted more fully herein, the hinge 16 biases the hatch 14 closed against the frame 12 in a normally closed position such that liquid flowing through the lumen 34 of the vessel 30 cannot escape through the percutaneous access device 10.

Referring specifically to FIG. 2, an object 50 (e.g., a needle), passes through the skin 40 and passes towards the percutaneous access device 10. Upon the application of force against the hatch 14 from the object 50, e.g., that extends through the passageway 12A (i.e., through the frame 12 and the extension 20), the hatch 14 hingedly swings open outwardly from the frame 12 and inwardly towards the lumen 34 of the vessel 30. This allows the object 50 to extend through the passageway 12A and pass into the lumen 34 of the vessel 30. Moreover, the hinge 16 biases the hatch 14 back to the normally closed position (as illustrated in FIG. 1) upon removal of the object 50 (e.g., the needle).

For instance, a user can run a finger over the layer of skin 40 until the alignment protrusion 22 is felt under the skin. Using the alignment protrusion 22 as a guide, a user inserts a needle through the layer of skin 40 and into the passageway 12A defined by the frame 12. The tip of the needle presses against the hatch 14 until sufficient force is applied by the user to pivot the hatch (via the hinge 16) from the normally closed position, to a hingedly open. In this manner, the hatch 14 opens inwardly into the vessel 30. Once the hatch 14 opens, the needle can extend into the lumen 34 of the vessel 30 so that fluid can be extracted or injected into the vessel 30. In this regard, an O-ring or other sealing mechanism can be utilized to seal any gap that may exist between the outside diameter of the needle and the inside diameter of the hatch 14.

Example Percutaneous Access Device

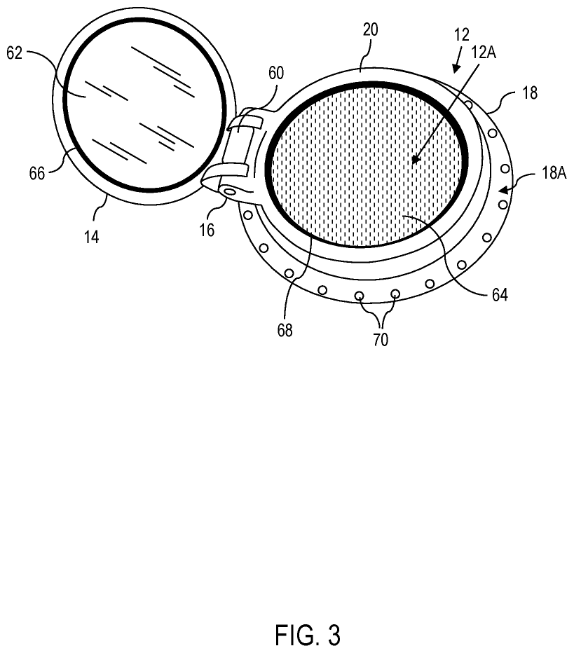

Referring to FIG. 3, an example percutaneous access device 10 is illustrated. The percutaneous access device 10 of FIG. 3 can be utilized to implement the percutaneous access device 10 of FIG. 1 and FIG. 2. As such, like elements are illustrated with like reference numbers. Where like reference numbers have previously been introduced, a description thereof is adopted by analogy to the corresponding preceding FIGURE(s). Accordingly, differences are discussed in detail and similarities are omitted to avoid repetition. The percutaneous access device 10 is illustrated flipped “upside down” for purposes of illustration of aspects of an exemplary implementation thereof.

As illustrated in the example embodiment of FIG. 3, the hatch 14, and correspondingly, the passageway 12A through the frame 12 (including the base member 18 and the extension 20), are generally round in cross-section. However, other shapes may be utilized. Moreover, the dimensions of the percutaneous access device 10 can vary, for instance, depending upon the size of the vessel to which the percutaneous access device 10 is to be attached. Moreover, the percutaneous access device 10 can be configured such that the passageway 12A is just slightly larger than the needles to be used for self-cannulation.

The percutaneous access device 10 can be made from any material or materials suitable for medical implantation in the body of a patient. For instance, the frame 12, the hatch 14, the hinge 16, components thereof, sections thereof, combinations thereof etc., may be titanium, stainless steel, medical grade silicone etc. Thus, in some embodiments, the percutaneous access device 10 can include at least one of a titanium material or a stainless steel material.

In the illustrative implementation, the hinge 16 includes a spring 60 (e.g., which may also be titanium, stainless steel or other material) that biases the hatch 14 into the normally closed position with respect to the frame 12. When an object such as a needle urges against a major surface 62 of the hatch 14, the spring 60 resists the hatch 14 from opening away from the frame 12. However, the force is overcome by the needle to open the hatch 14 sufficiently for self-cannulation. When the needle is removed from the percutaneous access device 10, the spring 60 biases the hatch 14 back to its normally closed position. As illustrated in FIG. 3, the hatch 14 is propped open away from the frame 12 solely for clarity of discussion of the parts thereof. Normally, with no object present, the hatch 14 would be biased closed against the frame 12, e.g., by the spring 60. In some embodiments, the hinge 16 is oriented such that a normal flow of a fluid through the vessel can assist in closing the hatch 14 when the object is removed from the percutaneous access device 10. Likewise, the normal flow of the fluid can also assist in keeping the hatch 14 closed against the frame 12.

In certain illustrative implementations, an optional self-healing layer 64 may be provided, e.g., within the passageway 12A through the base member 18, extension 20 or both. Where utilized, the self-healing layer 64 is a self-healing material secured within the passageway 12A such that an object such as a needle inserted through the passageway penetrates the self-healing material to open the hatch 14 and access the lumen of the vessel, and retraction of the object causes the self-healing material to seal the passageway 12A to prevent fluids flowing in the lumen of the vessel to escape through the percutaneous access device 10. For instance, the self-healing layer 64 may comprise an elastomeric material. The passageway through the frame 12 can be configured to be just slightly larger than the needle selected for self-cannulation. However, even if tolerances allow gaps around the needle, the self-healing layer 64 can be utilized to prevent fluid, e.g., blood, from leaking around the needle and out the percutaneous access device 10 during use in receiving dialysis. For instance, a self-healing rubber or other suitable material may be implemented in the passageway through the frame 12 so that when the needle is inserted through the percutaneous access device 10, the self-healing material conforms around the exterior circumference of the needle, thus preventing any leakage through the portal opening between the needle, the hatch 14 and the passageway through the frame 12. Here, the self-healing layer 64 can be utilized in addition to, or in lieu of, O-ring(s), gasket(s), or other devices that seal around an object such as a needle inserted into the percutaneous access device 10.

In another illustrative example, the hatch 14 may optionally include a first gasket 66, such as an O-ring about the hatch 14, an optional second gasket, such as a second O-ring 68 within the passageway 12A, a first O-ring that mates with a corresponding optional second O-ring, e.g., which may be seated adjacent to the passageway at the opening of the extension 20, etc. The use of one or more of the first gasket 66 and second gasket 68, provides a seal to assist with preventing fluid from escaping through the percutaneous access device when not in use.

In practical applications, the percutaneous access device 10 may include any no sealing mechanism, or any one or more desired sealing mechanisms, such as one or more O-rings, a self-healing layer 64, a combination thereof, etc.

The base member 18 may also have at least one feature, e.g., suture receiving apertures 70 therethrough to facilitate surgical implantation of the percutaneous access device 10. As such, the percutaneous access device 10 comprises at least one feature provided on the frame 12 that facilitates suturing the percutaneous access device 10 to the vessel 30.

In certain illustrative implementations, the base member 18 may be contoured so as to generally or approximately conform to the cylindrical profile of the vessel to which the percutaneous access device 10 is intended to be attached. Thus, a side view of the percutaneous access device 10 along the longitudinal axis of the vessel 30 would exhibit an arcuate cross-section.

Example Percutaneous Access Port with Sleeve

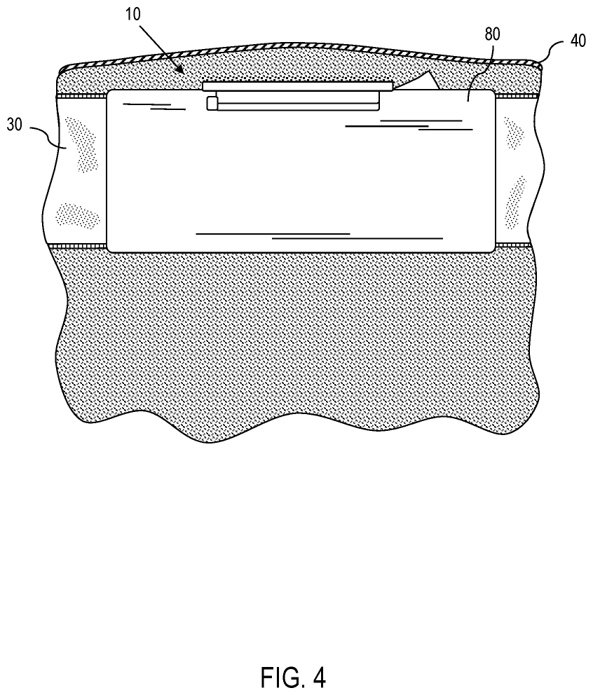

Referring to FIG. 4, a percutaneous access device 10 is illustrated according to further aspects of the present disclosure. The percutaneous access device 10 is analogous to the percutaneous access device 10 described with reference to FIG. 1-FIG. 3. As such, like structure is illustrated with like reference numbers, and the previous descriptions with reference to FIG. 1-FIG. 3 are adopted by analogy to FIG. 4, except where otherwise noted. As illustrated in FIG. 4, the percutaneous access device 10 differs from the percutaneous access device 10 of FIG. 1-FIG. 3 in that in FIG. 1-FIG. 3, the percutaneous access device 10 sutures directly to a vessel 30. By comparison, in FIG. 4, the percutaneous access device 10 includes a sleeve 80. The sleeve 80 secures around the vessel 30 and engages the base member 18 of the frame 12 when the percutaneous access device 10 is implanted in the patient. The sleeve 80 can be used in AV fistulas to improve the securement of the percutaneous access device 10 to the AV fistula. The sleeve 80 and corresponding percutaneous access device 10 can also replace a tube normally used in an AV graft.

In illustrative implementations, the sleeve 80 comprises a soft tissue patch, such as an inert biomaterial expanded polytetrafluoroethylene, e.g., ePTFE material (by Gore Tex Medical). Otherwise, the percutaneous access device 10 is substantially as described with reference to FIGS. 1-3. The sleeve 80 may facilitate connection of the percutaneous access device 10 to the vessel 30, e.g., AV fistula. For instance, the sleeve 80 can make it easier to suture the port to an AV fistula. Also, the frame 12 of the percutaneous access device 10 can be secured to the sleeve 80 where the sleeve 80 is sutured to the AV fistula. The sleeve 80 can be processed to include relatively harder and/or softer regions. For instance, the bottom of the sleeve can be hardened to resist puncture by a needle inserted into the vessel.

By way of example, surgical anastomosis can be created to connect the sleeve 80 to the vessel 30 using suture sewn by hand, mechanical staplers, biological glues, compression fitting techniques, etc. For instance, in an exemplary implementation, the sleeve 80 may be sutured to the vessel 30 along the edges thereof so as to stitch along the circumference of the vessel 30 at each end of the sleeve 80. This allows the port 10 to be grafted in-line with the vessel 30. Alternatively, the port 10 may be installed via an incision through the vessel 30 or using other suitable techniques.

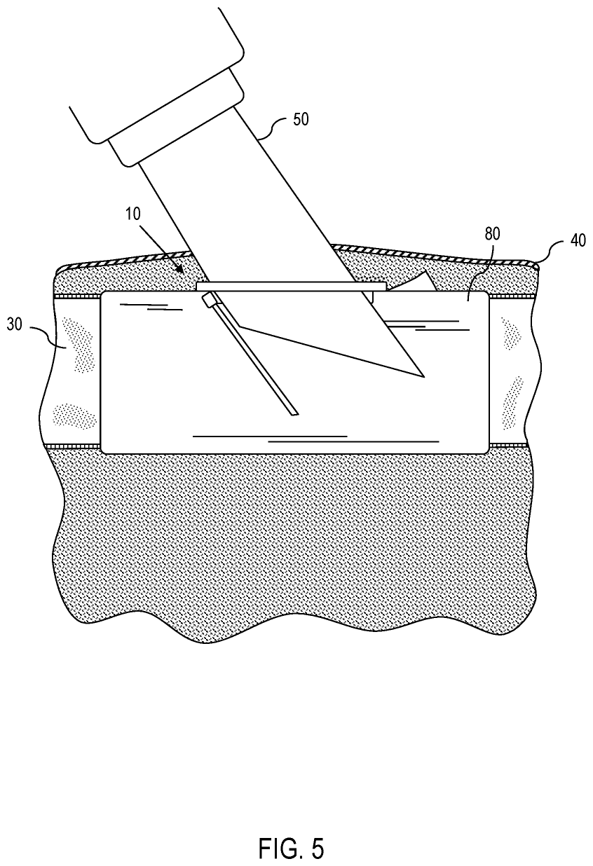

Referring to FIG. 5, the percutaneous access device 10 of FIG. 4 is illustrated in an operative position wherein an object 50, i.e., a needle, is inserted through the passageway to open the hatch 14. In certain illustrative implementations, the sleeve 80 can blanket over the percutaneous access device 10. As such, the sleeve 80 may serve to further prevent the likelihood of fluid leaking out of the passageway when the percutaneous access device is in use. The sleeve 80 may also comprise wraps or layers of material to define the sleeve. In this regard, the wraps can even cover over the frame 12 as a needle can puncture through the wrap.

As such, the percutaneous access device 10 described more fully herein is can be used as a vascular port that is suitable for use by patients that require dialysis. More particularly, the percutaneous access device 10 described more fully herein can be used as a “buttonhole port” that facilitates a self-cannulation method, allowing patients to insert their dialysis needles into the same spot each time they undergo dialysis. In the case of dialysis, there would likely be two percutaneous access devices 10 implanted, one to extract blood, and one to return the cleaned blood back into the patient's body. Alternatively, two sets of frame 12, hatch 14, and hinge 16 can be coupled to a corresponding sleeve. In this regard, the percutaneous access device 10 is suitable for use with any vascular bodily vessel, e.g., vein, artery, etc.

The hinge 16 can be any suitable structure that allows a needle to be repeatedly inserted and withdrawn into a patient in a manner that minimizes stresses and repeated puncturing to the body of the patient by the needle. For instance, as noted in greater detail herein, the hinge 16 may be and/or include a spring biased so that hatch 14 is normally urged closed against the frame 12. When a needle is inserted through the percutaneous access device 10, the hatch 14 hinges open to allow the needle to pass through the passageway and into the lumen of the vessel. When the needle is retracted, the spring of the hinge 16 biases the hatch 14 back to once again, close the hatch 14 against the frame 12.

More particularly, in a practical example, as someone cannulates the fistula through the percutaneous access device 10 using a needle used for dialysis, chemotherapy, etc., the hatch 14 opens inward allowing the needle to enter the blood stream. During this time, the self-healing material 64, gaskets, O-rings, sealer, combination thereof, etc. (where utilized) conforms around the needle in the passageway in the area of the frame 12, prohibiting bleeding through the percutaneous access device 10. After the treatment, the needle is pulled from the percutaneous access device 10 and the hatch 14 automatically closes due to the spring bias of the hinge 16. As such, the percutaneous access device 10 of the present disclosure closes the area from which the needle was pulled, which will keep the needle site from excessive post treatment bleeding.

In conventional dialysis, should a patient prematurely pull the needles from their insertion points, the flow through the dialysis machine will cause blood to spurt out of the patient, which can lead to excess blood loss. However, the port herein automatically closes, thus minimizing or eliminating blood loss.

Example Percutaneous Access Device with Body

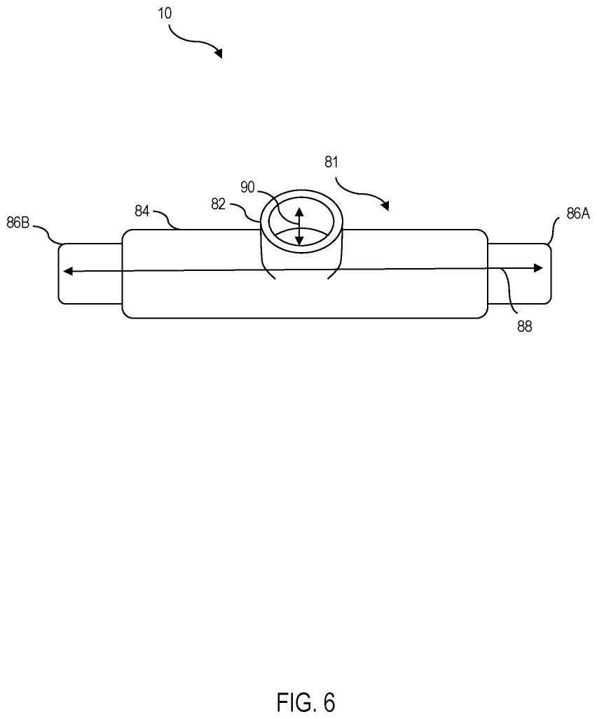

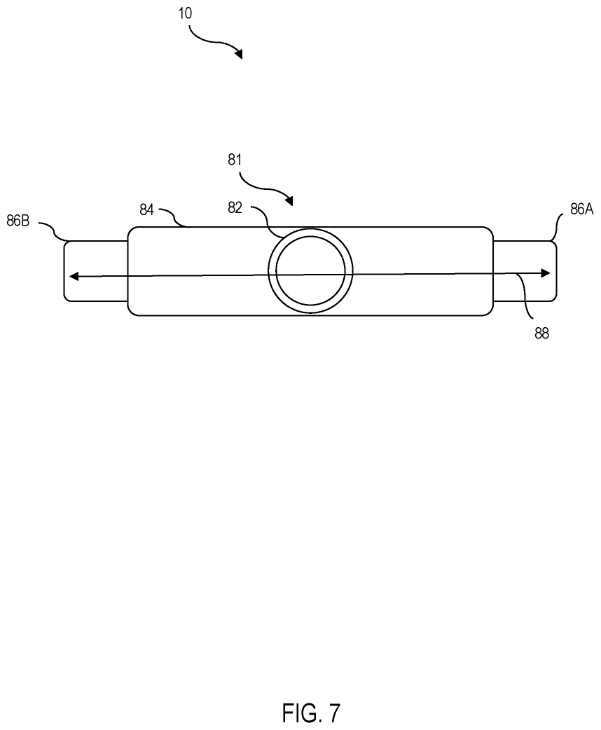

Referring to FIG. 6 and FIG. 7 generally, yet another percutaneous access device 10 is illustrated according to yet further aspects of the present disclosure. The percutaneous access device 10 is analogous to the percutaneous access device 10 described with reference to FIG. 1-FIG. 5. As such, like structure is illustrated with like reference numbers, and the previous descriptions with reference to FIG. 1-FIG. 5 are adopted by analogy to FIG. 6 and FIG. 7, except where otherwise noted. As illustrated in FIG. 6 and FIG. 7, the percutaneous access device 10 differs from the percutaneous access device 10 of FIG. 1-FIG. 5 in that the illustrated percutaneous access device 10 includes a body 81. By comparison, the percutaneous access device 10 of FIG. 1-FIG. 3 includes a frame 12 that sutures directly to a vessel 30, and the percutaneous access device 10 of FIG. 4-FIG. 5 includes a sleeve 80 that couples to a vessel.

Particularly, in FIG. 6 and FIG. 7, the percutaneous access device 10 includes a body 81 having an optional neck 82 that transitions into a shoulder 84. A set of inserts 86A, 86B (collectively, insert 86) project from opposing ends of the shoulder 84. For instance, as illustrated, a first insert 86A extends to the right of the shoulder 84 and a second insert 86B extends to the left of the shoulder 84. Notably, the inserts 86A, 86B form tube-like structures that meet with a hollow of the shoulder 84 so as to allow flow through the percutaneous access device.

Flow can also occur through the neck 82 when the hatch (not illustrated in FIG. 6 and FIG. 7 for sake of clarity) is open, as described more fully herein. That is, a first passageway 88 (e.g., a first continuous passageway) extends through the first insert 86A, through the shoulder 84 and through the second insert 86A. A second passageway 90 extends through the neck 82 and into the first passageway 88, e.g., within the main body at the shoulder 84. In FIG. 6 and in FIG. 7, the frame 12, hatch 14, and hinge 16 are not illustrated for purposes of clarity of illustration of the body 81.

The body 81 or any component thereof can be made of titanium, stainless steel, medical grade silicone, etc. Moreover, various components of the body 81 can have different hardness, depending upon the location. For instance, the shoulder 84 under the neck 82 can be hardened to resist puncture by a needle inserted too far into the vessel, etc. Correspondingly, the inserts 86 can be soft enough to be sutured to the vessel, etc.

In FIG. 6-FIG. 7, the frame 12, hatch 14, and hinge 16 are omitted for clarity of discussion to clearly illustrate the feature of the body 81. However, in practice, the hatch operates as described with reference to FIG. 1-FIG. 5, opening inward into the neck 82 towards the shoulder 84, opening inward into the shoulder 84, etc.

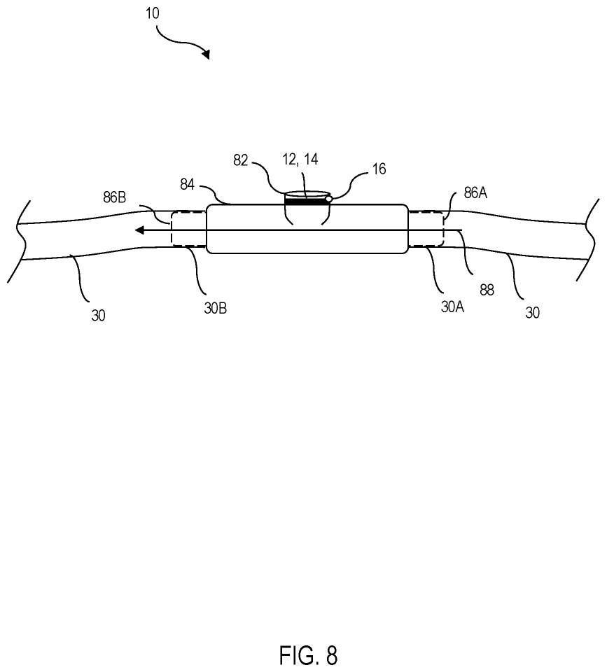

Referring to FIG. 8, the percutaneous access device 10 of FIG. 6 and FIG. 7 is illustrated installed in a vessel 30. In the embodiment of FIG. 8, the vessel 30 is cut so that each vessel end 30A, 30B extends over a corresponding insert 86A, 86B. Particularly, a first vessel end 30A of the vessel 30 is slipped over the first insert 86A, and is attached thereto. For instance, the first vessel end 30A can be coupled to the first insert 86A using sutures sewn by hand, mechanical staplers, biological glues, compression fitting techniques, etc. Likewise, a second vessel end 30B of the vessel 30 is slipped over the second insert 86B, and is attached thereto. Analogous to that above, the second vessel end 30B can be coupled to the second insert 86B using sutures sewn by hand, mechanical staplers, biological glues, compression fitting techniques, etc.

In addition to, or in lieu of the above, the body 81 or a portion thereof (e.g., the first insert 86A and second insert 86B) can be wrapped with a sleeve, wrapping, or other material (not shown for clarity). For instance, a sleeve such as the sleeve 80 (FIG. 5) can be incorporated over the vessel 30, inserts 86A, 86B, shoulder 84, neck 82, or a combination thereof. For instance, a soft tissue patch, such as an inert biomaterial expanded polytetrafluoroethylene, can be used to wrap over the vessel 30 at the connection to the inserts 86A, 86B, thus ensuring that the vessel walls are supported both internally (via the inserts 86A, 86B) and externally (via the wrap, sleeve, slip, etc.) at the port site.

As illustrated in FIG. 8, assume that blood is flowing through a vessel 30 from right to left. Here, the first passageway 88 includes a single arrowhead to also designate flow direction. The frame 12, hatch 14, and hinge 16 are illustrated positioned within the neck 82. The hatch 14 is hinged so as to be able to pivot downward through the neck 82 towards and/or into the shoulder 84 when an object such as a needle is inserted into the second passageway 90. Here, the hinge 16 is positioned “upstream” of the flow direction through the first passageway 88. As such, the flow can assist in closing the hatch 14 when the object (e.g., needle) is removed.

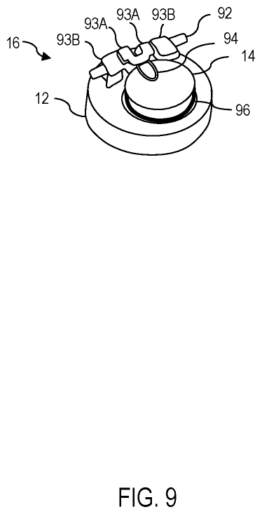

Referring to FIG. 9, a frame 12, a hatch 14, and a hinge 16 define an assembly that can be fitted into the neck 82 (FIG. 6-FIG. 8). In the view shown, the hatch 14 is flipped upside down. In practice, the assembly can be press fit, glued, mechanically fastened, mechanically locked, or otherwise coupled to the neck 82 (FIG. 6-FIG. 8).

As illustrated, the assembly includes an annular, ring-shaped frame 12 having a hollow therethrough. A major surface of the frame 88 includes a hinge 16 and a hatch 14 thereon. Because of the hinge 16, the hatch 14 is offset from the center of the annular frame 12, but still aligns over the hollow so that when the hatch 14 is hinged open, a passageway is present through the hatch 14. In practical embodiments, the frame 12 can be positioned in-line with the neck 82.

The hinge 16 includes a hinge pin 92 that passes through one or more knuckles 93A of the hinge 16 and corresponding knuckles 93B of the hatch 14. The hinge pin 92 cooperates with the knuckles 93A and knuckles 93B to enable the hatch 14 to pivot relative to the frame 12, e.g., to transition from open to closed positions. A spring 94 cooperates with the hinge 16 to apply a spring force against the hatch 14, e.g., via a loop of the spring 94 as shown to bias the hatch 14 closed against the frame 12. In some embodiments, the spring 94 can be encapsulated or otherwise sealed against the hatch 14, frame 12, hinge 16, or combination thereof.

In the illustrated embodiment, the hatch 14 can also include one or more O-rings 96. For instance, there can be an “outer” O-ring 96 around the outside perimeter of the hatch 14 or otherwise positioned between the hatch 14 and the frame 12 when the hatch 14 is closed. As another example, there can be an O-ring 96 on the inside diameter of the annular frame 12. The use of a pair of O-rings can be utilized to ensure that there is no flow through the hatch 14 when the hatch 14 is biased closed, and/or to ensure that there is no loss of vessel fluid (e.g., blood) through the hatch 14 when a needle is inserted through the hatch and into the lumen of the vessel 30. Analogous to that described more fully herein, other gasket, sealant, and other arrangements can also/alternatively be used, including the use of a self-healing material, as described more fully herein.

With reference to FIG. 6-FIG. 9 generally, in practical applications, the configuration of the frame 12, hatch, 14, and hinge 16 described with reference to FIG. 9 can be utilized with any of the embodiments herein. Likewise, the body of 81 described with reference to FIG. 6-FIG. 8 can utilize any of the other frame 12, hatch 14, and hinge 16 configurations described herein.

Percutaneous Access Device with Alignment Fixture

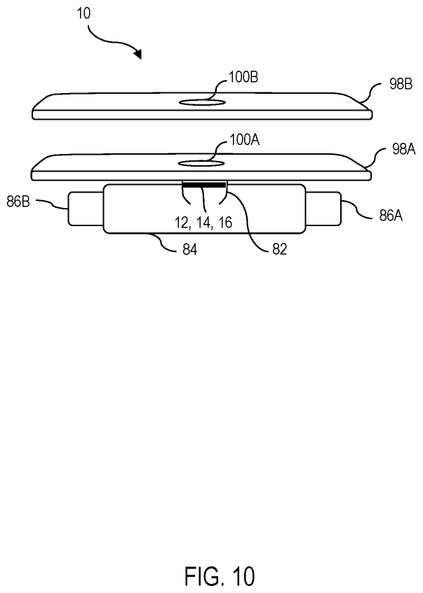

Referring to FIG. 10, yet another percutaneous access device 10 is illustrated. Here, the percutaneous access device 10 can be implemented in a manner analogous to any of the configurations described more fully herein, except that the percutaneous access device 10 further includes a subcutaneous alignment fixture 98A that cooperates with an optional, corresponding supercutaneous alignment fixture 98B. As such, the preceding description of components is adopted by analogy to like elements.

For sake of clarity of discussion, the illustrated subcutaneous access device 10 is implemented in a manner analogous to that described with reference to FIG. 6-FIG. 9, with the addition of a subcutaneous alignment fixture 98A coupled to the neck 82. For instance, as illustrated, the subcutaneous alignment fixture 98A is implemented as a plate that extends from the neck 82 and includes an aperture 100A that aligns with the aperture through the neck 82, thus forming a passageway into the shoulder 84. However, the subcutaneous alignment fixture 98A can also/alternatively be integrated into other embodiments described more fully herein. In some embodiments, the percutaneous access device 10 can mate with a corresponding supercutaneous alignment fixture 98B.

More particularly, the subcutaneous alignment fixture 98A includes an aperture 100A that aligns with the neck 82 and correspondingly, the second passageway. The supercutaneous alignment fixture 98B includes an aperture 100B therethrough. In some applications, the aperture 100B is the same size or smaller than the aperture 100A. As such, when the supercutaneous alignment fixture 98B is properly aligned with the subcutaneous alignment fixture 98A, the aperture 100B aligns with aperture 100A to assist a patient or a clinician in finding the hatch 14 of the percutaneous access device 10, as will be described in greater detail herein.

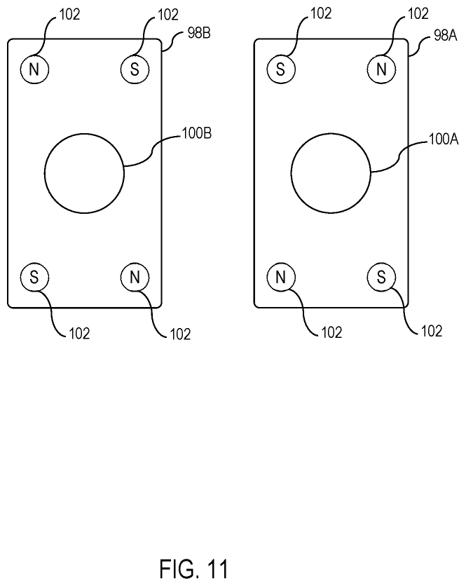

Referring briefly to FIG. 11, in an example embodiment, the subcutaneous alignment fixture 98A and the supercutaneous alignment fixture 98B include an automatic alignment feature, e.g., magnets as shown. For instance, as illustrated, the subcutaneous alignment fixture 98A and the supercutaneous alignment fixture 98B are each implemented as a plate. Moreover, the subcutaneous alignment fixture 98A includes an aperture 100A therethrough generally centered in the plate as illustrated. Likewise, the supercutaneous alignment fixture 98B includes an aperture 100B therethrough, also generally centered in the plate as illustrated. Each corner of the plate includes mating magnets 102 that will cause the plates to align together when the supercutaneous alignment fixture 98B is brought into close range/alignment of the subcutaneous alignment fixture 98A. Thus, the plates will automatically “snap” into proper alignment due to the magnetic attraction of the magents. When the plates align, the apertures 100A, 100B correspondingly align.

In an example application, the magnets 102 include surgical grade neodymium magnets. In practical applications, the positioning and the location of the magnets 102 can vary so long as the aperture of the supercutaneous alignment fixture 98B aligns with the aperture of the subcutaneous alignment fixture 98A. Moreover, the magnets 102 need not be exposed. For instance, where the percutaneous access device 10 is made of silicone, the magnets 102 can be encapsulated in the subcutaneous alignment fixture 102A and/or supercutaneous alignment fixture 102B. Alternatively, the magnets can be seated in pockets, glued in, mechanically fastened in place, etc. Moreover, since the supercutaneous access fixture 102B is separate from the percutaneous access device 10, the supercutaneous access fixture 102B can be a different material, e.g., plastic, rubber, metal, etc.

Purely by way of illustration, the subcutaneous alignment fixture 98A includes four magnets 102, including a south polarity oriented magnet in the upper left hand corner, a north polarity oriented magnet in the upper right hand corner, a south polarity oriented magnet in the lower right hand corner, and a north polarity oriented magnet in the lower left hand corner.

Correspondingly, the supercutaneous alignment fixture 98B includes four magnets, including a south polarity oriented magnet in the upper right hand corner, a north polarity oriented magnet in the upper left hand corner, a south polarity oriented magnet in the lower left hand corner, and a north polarity oriented magnet in the lower right hand corner. As such, when the supercutaneous alignment fixture 98B is “aligned” with the subcutaneous alignment fixture 98A, the opposite polarity magnets attract, precisely aligning the aperture in the supercutaneous alignment fixture 98B with the aperture in the subcutaneous alignment fixture 98A.

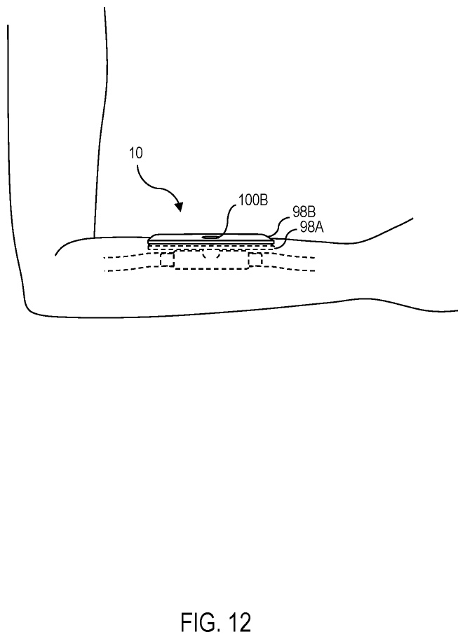

Referring to FIG. 12, the percutaneous access device 10 is installed in the arm of a patient, and this thus schematically illustrated in dashed lines. When the clinician or patient needs to insert a needle into the port, the individual lowers the supercutaneous fixture plate 98B over the skin of the arm. When the magnets come into sufficient proximity, the opposite polarity magnets “snap” the supercutaneous alignment fixture 98B to the subcutaneous alignment fixture 98A, thus providing the individual a visual guide above the skin, where to insert a needle to perfectly align with the hatch 14, and thus, to insert the needle into the port.

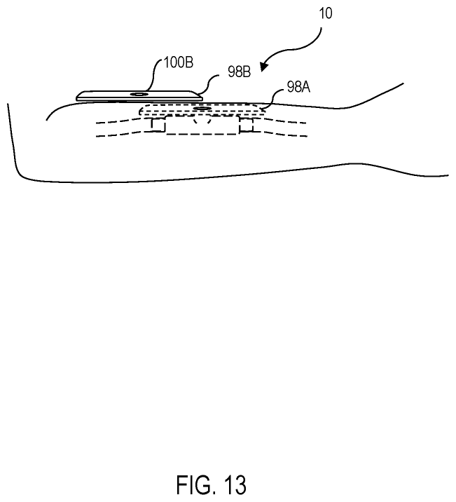

Referring to FIG. 13, when the procedure is complete, the supercutaneous fixture plate 98B is freed from the subcutaneous fixture plate e.g., by shearing the magnets apart.

Two Port Percutaneous Access Device

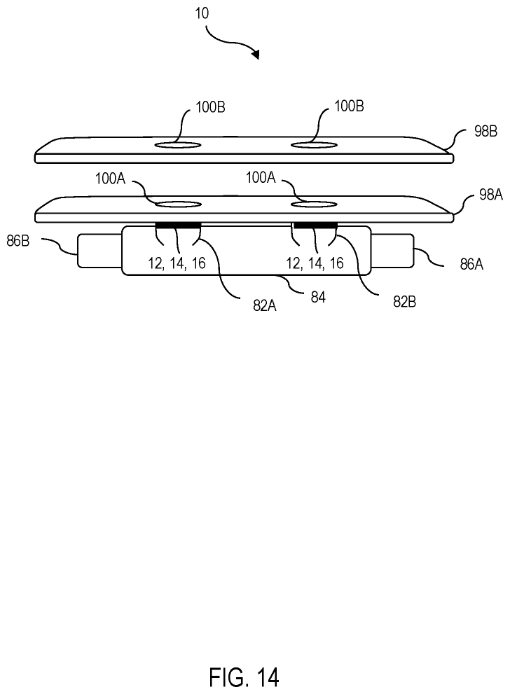

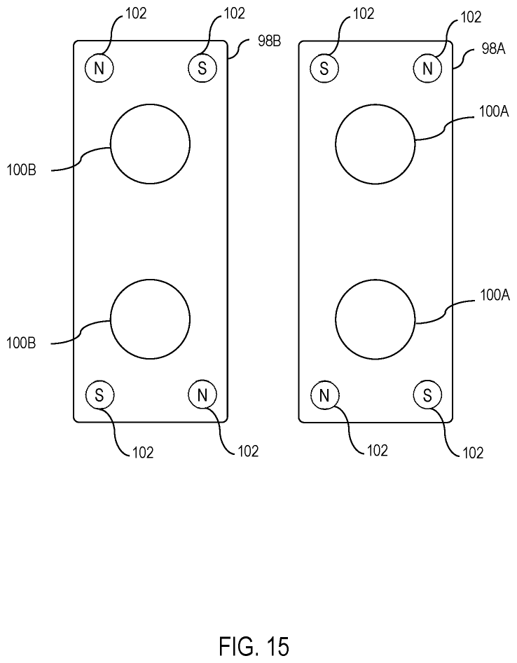

With reference to FIG. 14 and FIG. 15, the percutaneous access device 10 can be expanded to include multiple hatches, e.g., for dialysis, etc. The embodiment of FIGS. 14 and 15 is identical to the embodiment of FIG. 10-FIG. 13 except that the subcutaneous access device 10 includes two necks 82, and one assembly in each neck 82 including the frame 12, hatch 14, and hinge 16, as described more fully herein.

Moreover, as illustrated, the subcutaneous alignment fixture 98A and the supercutaneous alignment fixture 98B each include two apertures, arranged to align with the corresponding necks 82.

For instance, the percutaneous access device 10 includes a first neck 82A that transitions into a shoulder 84, and a second neck 82B that transitions into the shoulder 84. Also, a first insert 86A extends from a first end of the shoulder 84, and a second insert 86B extends from a second end of the shoulder 84. In this regard, a first passageway extends through the first insert 86A, through the shoulder 84, and through the second insert 86B. A second passageway extends through the first neck 82A and into the first passageway. A third passageway extends through the second neck 86B and into the first passageway. A first frame 12 has a portal opening therethrough, where the first frame 12 is in-line with the first neck 82A. A first hatch 14 is hingedly coupled to the first frame 12 such that the first hatch 14 is biased in a normally closed position against the first frame 12 as described more fully herein. Analogously, a second frame 12 has a portal opening therethrough, and is in-line with the second neck 82B. Here, a second hatch 14 is hingedly coupled to the second frame 12 such that the second hatch 14 is biased in a normally closed position against the second frame 12 in the second neck 82B.

Analogous to that described more fully herein, when the percutaneous access device implanted in a patient, the first frame and the second frame are positioned beneath a layer of skin of the patient. The first hatch is biased closed against the first frame (in the first neck 82A) in a normally closed position such that liquid flowing through a lumen of the vessel cannot escape through the percutaneous access device. Likewise, the second hatch is biased closed against the second frame (in the second neck 82B) in a normally closed position such that liquid flowing through a lumen of the vessel cannot escape through the percutaneous access device. However, the application of force against the first hatch from a first object outside the skin (e.g., a first needle) causes the first hatch to open outwardly from the first frame and inwardly towards the shoulder (i.e., towards the lumen of the vessel). The first hatch biases back to the normally closed position upon removal of the first object. Likewise, the application of force against the second hatch from a second object outside the skin (e.g., a second needle) causes the second hatch to open outwardly from the second frame and inwardly towards the shoulder (i.e., towards the lumen of the vessel). The second hatch biases back to the normally closed position upon removal of the second object. Here, the first hatch and second hatch are operable independent of each other.

As noted above, the percutaneous access device 10 can also include a subcutaneous alignment fixture coupled to the first neck and the second neck. Here, a supercutaneous alignment fixture aligns above the skin with the subcutaneous alignment fixture when the percutaneous access device is surgically installed in a patient.

In some embodiments, the subcutaneous alignment fixture includes at least one magnet, and the supercutaneous alignment fixture includes at least one magnet. For instance, as best illustrated in FIG. 15, analogous to the example embodiment described with reference to FIG. 11, and purely by way of illustration, the subcutaneous alignment fixture 98A includes four magnets 102, including a south polarity oriented magnet in the upper left hand corner, a north polarity oriented magnet in the upper right hand corner, a south polarity oriented magnet in the lower right hand corner, and a north polarity oriented magnet in the lower left hand corner.

Correspondingly, the supercutaneous alignment fixture 98B includes four magnets, including a south polarity oriented magnet in the upper right hand corner, a north polarity oriented magnet in the upper left hand corner, a south polarity oriented magnet in the lower left hand corner, and a north polarity oriented magnet in the lower right hand corner. As such, when the supercutaneous alignment fixture 98B is “aligned” with the subcutaneous alignment fixture 98A, the opposite polarity magnets attract, precisely aligning the aperture in the supercutaneous alignment fixture 98B with the aperture in the subcutaneous alignment fixture 98A.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A percutaneous access device, comprising:
a frame having a passageway that extends through the frame;
a hatch; and
a hinge that couples the frame to the hatch;
wherein:
when the percutaneous access device implanted in a patient:
the frame is positioned beneath a layer of skin of the patient proximate a bodily vessel;
the hinge biases the hatch closed against the frame in a normally closed position such that liquid flowing through a lumen of the vessel cannot escape through the percutaneous access device;
the application of force against the hatch from an object that extends from outside the skin causes the hatch to open outwardly from the frame and inwardly towards the lumen of the vessel, allowing the object to extend through the passageway and pass into the lumen of the vessel; and
the hinge biases the hatch back to the normally closed position upon removal of the object.

2. The percutaneous access device according to claim 1, wherein:
the frame further comprises:
a base member having a first major surface; and
an extension that projects from the first major surface;
wherein:
the passageway extends through the base member and the extension;
wherein:
the base member is positioned beneath a layer of skin of the patient;
the first major surface of the base member faces the vessel; and
the extension passes through a wall of the vessel.

3. The percutaneous access device according to claim 1, wherein the frame, hatch and hinge include at least one of a titanium material or a stainless steel material.

4. The percutaneous access device according to claim 1, wherein:
the hinge includes a spring that biases the hatch into the normally closed position.

5. The percutaneous access device according to claim 1 further comprising:
a self-healing material secured within the passageway such that when the object, implemented as a needle, is inserted through the passageway, the needle penetrates the self-healing material to open the hatch and access the lumen of the vessel, and retraction of the needle causes the self-healing material to seal the passageway to prevent fluids flowing in the lumen to escape through the percutaneous access device.

6. The percutaneous access device according to claim 1 further comprising:
a sleeve that secures around the vessel and engages the base member when the percutaneous access device is implanted in the patient.

7. The percutaneous access device according to claim 6, wherein the sleeve comprises a soft tissue patch.

8. The percutaneous access device according to claim 6, wherein the sleeve comprises an inert biomaterial expanded polytetrafluoroethylene.

9. The percutaneous access device according to claim 1 further comprising:
an alignment protrusion that extends from the base member in a direction opposite the first major surface, the alignment protrusion indicating a known position beneath the skin relative to the passageway when the percutaneous access device is implanted in the patient.

10. The percutaneous access device according to claim 1 further comprising:
at least one feature provided on the frame that facilitates suturing the percutaneous access device to the vessel.

11. The percutaneous access device according to claim 10, wherein:
the at least one feature comprises the frame member having a plurality of apertures therethrough.

12. The percutaneous access device according to claim 1 further comprising:
a neck that transitions into a shoulder;
a first insert extending from a first end of the shoulder;
a second insert extending from a second end of the shoulder;
a first passageway extending through the first insert, through the shoulder, and through the second insert; and
a second passageway extending through the neck and into the first passageway.

13. The percutaneous access device according to claim 12, wherein:
the frame is implemented as an annular, ring-shaped frame having a hollow therethrough;
the hatch is hingedly coupled to the frame; and
the frame is positioned in-line with the neck.

14. The percutaneous access device according to claim 13 further comprising:
a first O-ring on an inside diameter of the frame; and
a second O-ring positioned between the hatch and the frame when the hatch is closed.

15. The percutaneous access device according to claim 12 further comprising:
a subcutaneous alignment fixture coupled to the neck; wherein:
a supercutaneous alignment fixture aligns above the skin with the subcutaneous alignment fixture when the percutaneous access device is surgically installed in a patient.

16. The percutaneous access device according to claim 15, wherein:
the subcutaneous alignment fixture includes at least one magnet; and
the supercutaneous alignment fixture includes at least one magnet.

17. The percutaneous access device according to claim 12, wherein:
the neck defines a first neck;
the hatch defines a first hatch coupled to the first neck;
further comprising:
a second neck that extends into the shoulder; and
a second hatch coupled to the second neck.

18. A percutaneous access device comprising:
a first neck that transitions into a shoulder;
a second neck that transitions into the shoulder;
a first insert extending from a first end of the shoulder;
a second insert extending from a second end of the shoulder;
a first passageway extending through the first insert, through the shoulder, and through the second insert; and
a second passageway extending through the first neck and into the first passageway;
a third passageway extending through the second neck and into the first passageway;
a first frame having a portal opening therethrough, the first frame in-line with the first neck;
a first hatch hingedly coupled to the first frame such that the first hatch is biased in a normally closed position against the first frame;
a second frame having a portal opening therethrough, the second frame in-line with the second neck; and
a second hatch hingedly coupled to the second frame such that the second hatch is biased in a normally closed position against the second frame;
wherein:
when the percutaneous access device implanted in a patient:
the first frame and the second frame are positioned beneath a layer of skin of the patient;
the first hatch is biased closed against the first frame in a normally closed position such that liquid flowing through a lumen of the vessel cannot escape through the percutaneous access device;
the second hatch is biased closed against the second frame in a normally closed position such that liquid flowing through a lumen of the vessel cannot escape through the percutaneous access device;
the application of force against the first hatch from a first object outside the skin causes the first hatch to open outwardly from the first frame and inwardly towards the shoulder;
the first hatch biases back to the normally closed position upon removal of the first object;
the application of force against the second hatch from a second object outside the skin causes the second hatch to open outwardly from the second frame and inwardly towards the shoulder;
the second hatch biases back to the normally closed position upon removal of the second object; and
the first hatch and second hatch are operable independent of each other.

19. The percutaneous access device according to claim 18 further comprising:
a subcutaneous alignment fixture coupled to the first neck and the second neck;
wherein:
a supercutaneous alignment fixture aligns above the skin with the subcutaneous alignment fixture when the percutaneous access device is surgically installed in a patient.

20. The percutaneous access device according to claim 19, wherein:
the subcutaneous alignment fixture includes at least one magnet; and
the supercutaneous alignment fixture includes at least one magnet.
